(19) United States
(12) Patent Application Publication
(10) Pub. No.: US 2010/0004512 A1
(43) Pub. Date: Jan. 7, 2010

(54) COUPLING AND ENDOSCOPE SYSTEM HAVING THE SAME

(75) Inventor: Yoshiaki Sueoka, Tokyo (JP)

Correspondence Address: VOLPE AND KOENIG, P.C., UNITED PLAZA, SUITE 1600, 30 SOUTH 17TH STREET, PHILADELPHIA, PA, 19103, US

(73) Assignee: OLYMPUS MEDICAL SYSTEMS CORPORATION, Tokyo (JP)

(21) Appl. No.: 12/495,180

(22) Filed: Jun. 30, 2009

(30) Foreign Application Priority Data
Jul. 2, 2008 (JP) 2008-173569

(51) Int. Cl. A61B 1/06; H01R 13/62

(52) U.S. Cl. 600/178; 439/345

(57) ABSTRACT

An object is to provide a coupling that prevents connection failure of a scope and a light source device and that is compatible with existing scopes as well as to provide an endoscope system having the same. A coupling employed includes a columnar plug and a socket into which the plug is inserted and connected. The plug has engaging projections that are radially projected from and retracted into an outer circumferential surface thereof. The socket has a connection surface to which an end surface of the plug is abutted, a pressing member having a through-hole that is slightly larger than the outside diameter of the plug, a coil spring that urges the pressing member along an insertion direction of the plug toward the connection surface, and stopper members that stop the pressing member so as to push it in a direction opposite to a direction toward the connection surface until the engaging projections pass through the through-hole in the pressing member. The stopper members stop pushing the pressing member after the engaging projections pass through the through-hole in the pressing member.

### BACKGROUND OF THE INVENTION

1. Field of the Invention

The present invention relates to a coupling that connects a plug and a socket, and to an endoscope system having the same.

This application is based on Japanese Patent Application No. 2008-173569, the content of which is incorporated herein by reference.

2. Description of Related Art

For example, when a scope and a light source device in an endoscope system are connected, unevenness is produced at a connecting portion of the scope and the light source device due to the dimensional tolerances of the scope etc. This varies the optical path length from the light source to an incident portion of the scope, resulting in a problem that the intensity of illumination light emitted from an end of the scope is unstable. To counter this, a method in which the scope and the light source device are connected with a coupling that has coil springs on both the plug side and the socket side is disclosed in the related art (for example, refer to Japanese Unexamined Patent Application, Publication No. 2004-37984).

However, the technique disclosed in Japanese Unexamined Patent Application, Publication No. 2004-37984 requires all scopes to have a plug or a socket having a coil spring. Thus, there is a disadvantage in that existing scopes cannot be connected to a light source device having such a socket.

### BRIEF SUMMARY OF THE INVENTION

The present invention has been made in view of the above-described circumstances, and an object thereof is to provide a coupling that prevents connection failure of a scope and a light source device and that is compatible with existing scopes, as well as to provide an endoscope system having the same.

To achieve the above-described object, the present invention employs the following solutions.

A first aspect of the present invention is a coupling including a columnar plug and a socket into which the plug is inserted and connected. The plug has engaging projections that are radially projected from and retracted into an outer circumferential surface thereof. The socket has a connection surface to which an end surface of the plug is abutted, a pressing member having a through-hole that is slightly larger than the outside diameter of the plug, an urging member that urges the pressing member along an insertion direction of the plug toward the connection surface, and stopper members that stop the pressing member so as to push it in a direction opposite to a direction toward the connection surface in response to the insertion of the plug into the socket until the engaging projections pass through the through-hole in the pressing member. The stopper members stop pushing the pressing member after the engaging projections pass through the through-hole in the pressing member, and the pressing member that is no longer pushed urges the plug toward the connection surface via the engaging projections.

In the above-described coupling, when the plug is inserted into the socket, the stopper members stop the pressing member from moving toward the connection surface until the engaging projections provided around the outer circumferential surface of the plug pass through the through-hole in the pressing member provided in the socket. Thus, the pressing member pushes the engaging projections provided around the outer circumferential surface of the plug radially inward into the plug, whereby the plug can be easily inserted into the socket. After the engaging projections pass through the through-hole in the pressing member, the stopper members no longer stop the pressing member. Thus, the pressing member urged by the urging member is moved toward the connection surface along the insertion direction of the plug. The engaging projections having passed through the through-hole in the pressing member project radially outward from the plug and engage with the pressing member so as to be urged by the pressing member. Thus, the urging force applied to the engaging projections is transmitted to the plug to move the plug toward the connection surface. As a result, the end surface of the plug is pressed against the connection surface of the socket.

That is, this coupling makes it easy to connect the plug and the socket and enables the end surface of the plug to be securely pressed against the connection surface of the socket. By applying this coupling to, for example, the connecting portion of the scope and the light source device of an endoscope system, the light source device and the scope can be easily and securely connected. Furthermore, because the optical path length from the light source to the incident portion of the scope can be controlled, stable illumination light can be emitted onto an observation portion.

In the above-described coupling, the stopper members may be provided so as to be movable in a direction intersecting the insertion direction of the plug and have inclined surfaces with which the end of the plug is brought into contact, the inclined surfaces being inclined inward along the direction intersecting the insertion direction of the plug in a direction toward the connection surface, an edge of the end surface may push apart the inclined surfaces facing thereto and move the stopper members in the direction intersecting the insertion direction of the plug when the plug is inserted into the socket, and the inclined surfaces facing thereto being pushed apart may urge the pressing member in a direction opposite to the direction toward the connection surface by a force greater than the urging force of the urging member.

With this configuration, by inserting the plug into the socket, the end of the plug can be brought into contact with the inclined surfaces provided on the stopper members to move the stopper members, which are provided so as to be movable in the direction intersecting the insertion direction of the plug, outward to cause the stopper members to no longer stop the pressing member.

The above-described coupling may further include other urging members that urge the stopper members inward along the direction intersecting the insertion direction of the plug.

With this configuration, after the plug is pulled away from the socket, the other urging members urge the stopper members inward along the direction intersecting the insertion direction of the plug to bring them back to a position where they can stop the pressing member from moving toward the connection surface.

In the above-described coupling, the pressing member and the stopper members may be arranged such that the end of the plug is in contact with the inclined surfaces of the stopper members before the engaging projections pass through the through-hole, and the pressing member may have a tapered inner surface with which edges of the inclined surfaces of the stopper members are brought into contact, the tapered inner surface being inclined radially outward of the through-hole in the direction toward the connection surface.

With this configuration, before the engaging projections pass through the through-hole, the plug inserted into the socket moves the stopper members outward in the direction intersecting the insertion direction of the plug and brings the edges of the inclined surfaces of the stopper members into contact with the tapered inner surface of the pressing member. Thus, the pressing member can be moved in a direction opposite to the insertion direction of the plug. This can increase the insertion force of the plug and makes it easy to insert the plug into the socket.

A second aspect of the present invention is an endoscope system including any one of the above-described couplings; a light source device that emits illumination light; and a scope that is provided so as to be attachable to and separable from the light source device and that guides the illumination light from the light source device to an observation area. The plug is a light guide member provided in the scope, the socket is provided in the light source device, the connection surface is an emitting surface of the illumination light from the light source device, and the end surface of the plug is an incident surface of the light guide member.

This endoscope system enables the scope and the light source device to be easily connected and the incident surface of the light guide member provided in the scope to be securely pressed against the emitting surface of the illumination light from the light source device, and also enables the optical path length from the light source to the incident portion of the scope to be controlled. Thus, it is possible to emit stable illumination light onto the observation area. In addition, the provision of the socket on the light source device makes connection to existing scopes possible.

The present invention has advantages in that it can prevent connection failure of the scope and the light source device and that it can be connected to an existing scope.

### BRIEF DESCRIPTION OF THE SEVERAL VIEWS OF THE DRAWINGS

FIG. 1 schematically shows the structure of a coupling according to an embodiment of the present invention.

FIG. 2 is a diagram for explaining a state in which a plug of the coupling in FIG. 1 is being inserted into a socket.

FIG. 3 is a diagram for explaining a state in which the plug of the coupling in FIG. 1 is being inserted into the socket.

FIG. 4 is a diagram for explaining a state in which the plug of the coupling in FIG. 1 is being inserted into the socket.

FIG. 5 is a diagram for explaining a state in which the plug of the coupling in FIG. 1 is completely connected to the socket.

FIG. 6 is a diagram for explaining a state in which the plug of the coupling in FIG. 1 is being pulled away from the socket.

FIG. 7 is a diagram for explaining a state in which the plug of the coupling in FIG. 1 is being pulled away from the socket.

FIG. 8 is a diagram for explaining a state in which the plug of the coupling in FIG. 1 is being pulled away from the socket.

FIG. 9 is a schematic view for explaining the structure of a light source device shown in FIG. 10.

FIG. 10 schematically shows the structure of an endoscope system according to an example application of the present invention.

FIG. 11 is a diagram for explaining a state in which the plug of the endoscope system in FIG. 10 is being inserted into the socket.

FIG. 12 is a diagram for explaining a state in which the plug of the endoscope system in FIG. 10 is being inserted into the socket.

FIG. 13 is a diagram for explaining a state in which the plug of the endoscope system in FIG. 10 is completely connected to the socket.

FIG. 14 is a diagram for explaining a state in which the plug of the endoscope system in FIG. 10 is being pulled away from the socket.

FIG. 15 is a diagram for explaining a state in which the plug of the endoscope system in FIG. 10 is being pulled away from the socket.

FIG. 16 is a diagram for explaining a state in which the plug of the endoscope system in FIG. 10 is being pulled away from the socket.

### DETAILED DESCRIPTION OF THE INVENTION

A coupling according to an embodiment of the present invention will be described below with reference to the drawings.

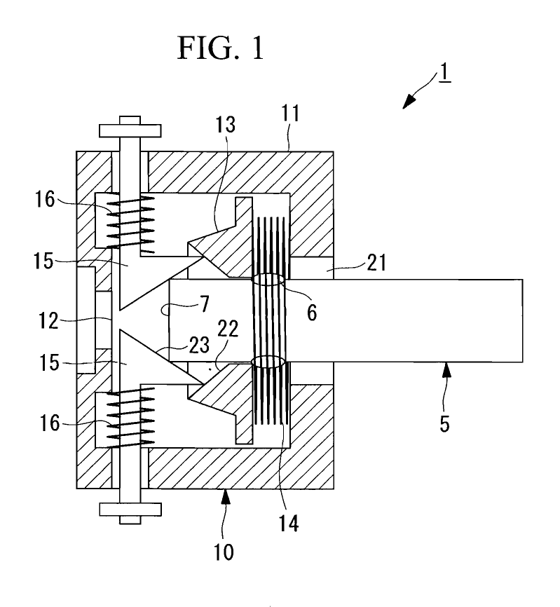

As shown in FIG. 1, the coupling 1 according to this embodiment includes a columnar plug 5 and a socket 10 into which the plug 5 is inserted and connected.

The plug 5 has a plurality of engaging projections 6 that are provided at intervals around the circumferential direction thereof and urged radially outward by springs (not shown) so as to be radially projectable from and retractable into the outer circumferential surface.

The socket 10 includes a housing 11 having an insertion hole 21 through which the plug 5 is to be inserted; a pressing member 13 having a through-hole for allowing the plug 5 to extend therethrough, the center of the through-hole being located on the central axis of the insertion hole 21; a coil spring (urging member) 14 disposed between the pressing member 13 and the inner surface of the housing 11 where the insertion hole 21 is provided; stopper members 15 provided so as to be movable in a direction intersecting the insertion direction of the plug 5; and coil springs (other urging members) 16 provided between the stopper members 15 and the inner circumferential surface of the housing 11.

The housing 11 is, for example, a cylindrical case that accommodates the pressing member 13, the coil spring 14, the stopper members 15, and the coil springs 16. A connection surface 12 to which an end surface 7 of the plug 5 is to be abutted is provided on the inner surface of the housing 11, opposite the inner surface of the housing 11 where the insertion hole 21 is provided.

The pressing member 13 is a circular member having a through-hole with a diameter that is slightly larger than the outside diameter of the plug 5 and is slightly smaller than a circle circumscribing ends of the engaging projections 6 in a projected state. By pushing the engaging projections 6 radially inward into the plug 5, the plug 5 can pass through the through-hole in the pressing member 13. By allowing the engaging projections 6 to project radially outward, the engaging projections 6 and the pressing member 13 are engaged. The pressing member 13 has a tapered inner surface 22 with which the stopper members 15 are brought into contact, the tapered inner surface 22 being inclined radially outward of the through-hole in the direction toward the connection surface 12.

The stopper members 15 are, for example, rod-shaped or plate-shaped members against which the pressing member 13 is abutted until the engaging projections 6 pass through the through-hole in the pressing member 13, so as to stop the pressing member 13 from moving toward the connection surface 12. The stopper members 15 have inclined surfaces 23 with which the end of the plug 5 is brought into contact, the inclined surfaces 23 being inclined inward along a direction perpendicular to the insertion direction of the plug 5, that is, radially inward of the housing 11, in the direction toward the connection surface 12. The stopper members 15, which are urged radially inward by the coil springs 16, are arranged at positions where the inclined surfaces 23 thereof are in contact with the end of the plug 5 before the engaging projections 6 pass through the through-hole in the pressing member 13.

That is, when the plug 5 is inserted into the socket 10, the end of the plug 5 pushes the inclined surfaces 23 provided on the stopper members 15. Thus, the stopper members 15 are moved radially outward. After the engaging projections 6 pass through the through-hole in the pressing member 13, the stopper members 15, having been moved radially outward, are moved radially outward from the outer circumferential surface of the pressing member 13 and release the pressing member 13 having stopped the movement of the plug 5 in the insertion direction.

The coil spring 14 urges the pressing member 13 toward the connection surface 12 along the insertion direction of the plug 5.

The coil springs 16 urge the stopper members 15 radially inward.

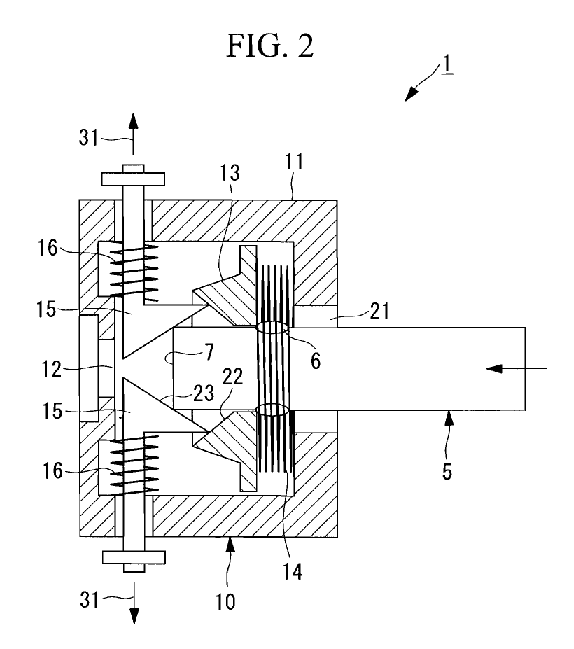

Referring to FIGS. 2 to 5, the operation of inserting the plug 5 into the socket 10 in the coupling 1 having the above-described structure will be described below.

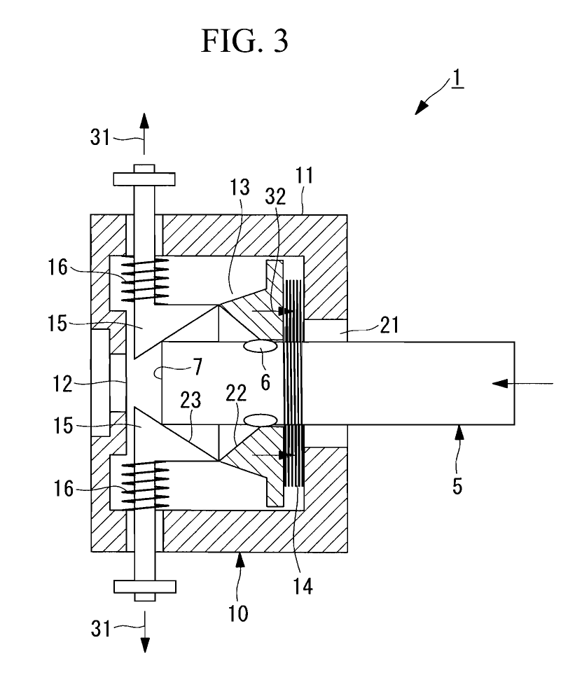

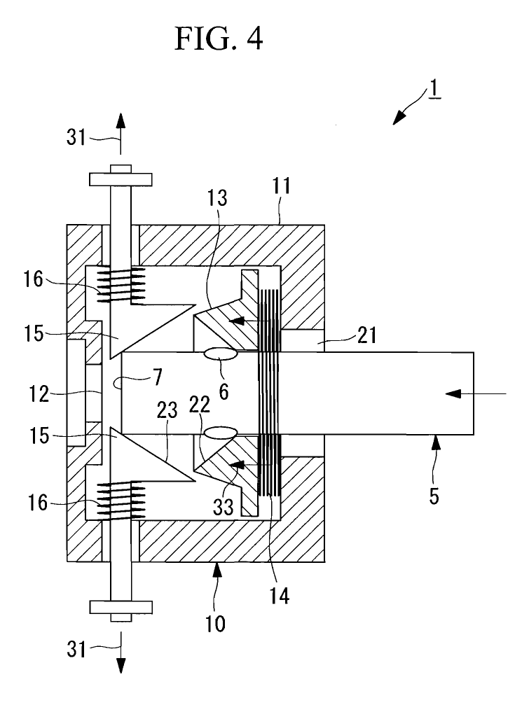

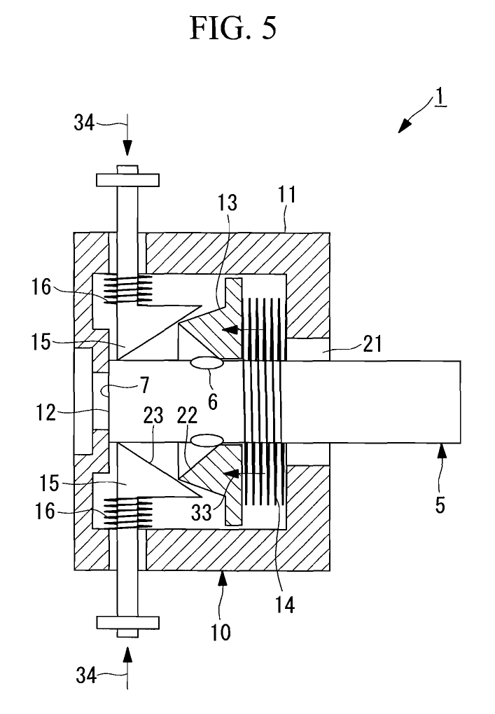

FIGS. 2 to 4 show states during insertion of the plug 5 into the socket 10. FIG. 2 shows a state before the engaging projections 6 pass through the through-hole in the pressing member 13, FIG. 3 shows a state in which the engaging projections 6 are passing through the through-hole in the pressing member 13, and FIG. 4 shows a state after the engaging projections 6 have passed through the through-hole in the pressing member 13. FIG. 5 shows a state in which the plug 5 is completely connected to the socket 10.

First, as shown in FIG. 2, when the plug 5 is inserted into the socket 10, the end of the plug 5 comes into contact with the inclined surfaces 23 of the stopper members 15, moving the stopper members 15 radially outward in the housing 11, i.e., in directions shown by arrows 31.

Then, as shown in FIG. 3, when the plug 5 is deeply inserted into the socket 10, the stopper members 15 are moved farther in the directions shown by the arrows 31. As a result, the stopper members 15 slide on the tapered inner surface 22 of the pressing member 13 and move the pressing member 13 in a direction opposite to the insertion direction of the plug 5, i.e., a direction shown by an arrow 32. This increases the insertion force of the plug 5 and causes the pressing member 13 to press the engaging projections 6 provided around the outer circumferential surface of the plug 5 radially inward into the plug 5. Thus, the plug 5 can be easily inserted into the socket 10.

Then, as shown in FIG. 4, when the plug 5 is more deeply inserted into the socket 10, the stopper members 15 are moved farther in the directions shown by the arrows 31, and the engaging projections 6 pass through the through-hole in the pressing member 13. At this time, by moving the stopper members 15 in the directions shown by the arrows 31 to positions where they do not touch the pressing member 13, the stopper members 15 no longer stop the pressing member 13. After passing through the through-hole in the pressing member 13, the engaging projections 6 provided around the outer circumferential surface of the plug 5 project radially outward from the plug 5 and engage with the pressing member 13.

Then, as shown in FIG. 5, when the stopper members 15 no longer stop the pressing member 13, the coil spring 14 moves the pressing member 13 along the insertion direction of the plug 5 toward the connection surface 12, i.e., in a direction shown by an arrow 33.

The urging force of the coil spring 14 is transmitted to the plug 5 via the pressing member 13 and the engaging projections 6, and the urging force moves the plug 5 toward the connection surface 12. Thus, the end surface 7 of the plug 5 is pressed against the connection surface 12 of the socket 10.

The stopper members 15 are urged in the inward radial direction of the plug 5, i.e., in directions shown by arrows 34, by the coil springs 16. By uniformly urging the plug 5 in the inward radial direction of the plug 5 with the plurality of stopper members 15, the end surface 7 of the plug 5 and the connection surface 12 of the socket 10 are aligned.

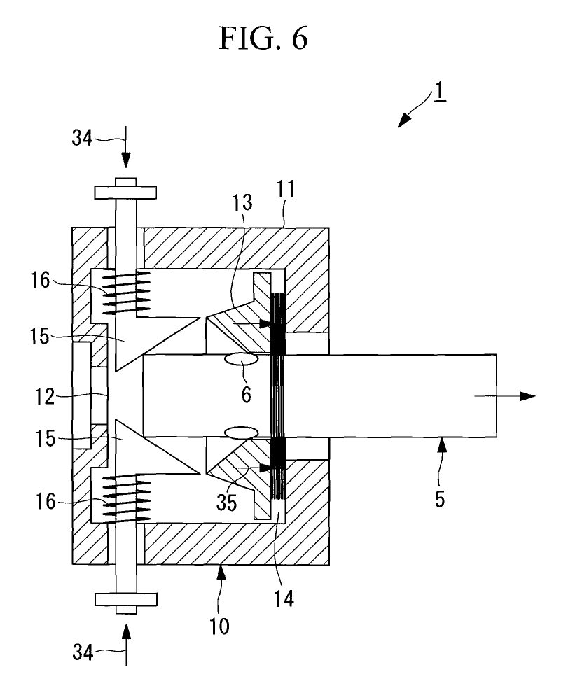

Referring to FIGS. 6 to 8, the operation of removing the plug 5 from the socket 10 in the coupling 1 having the above-described structure will be described below.

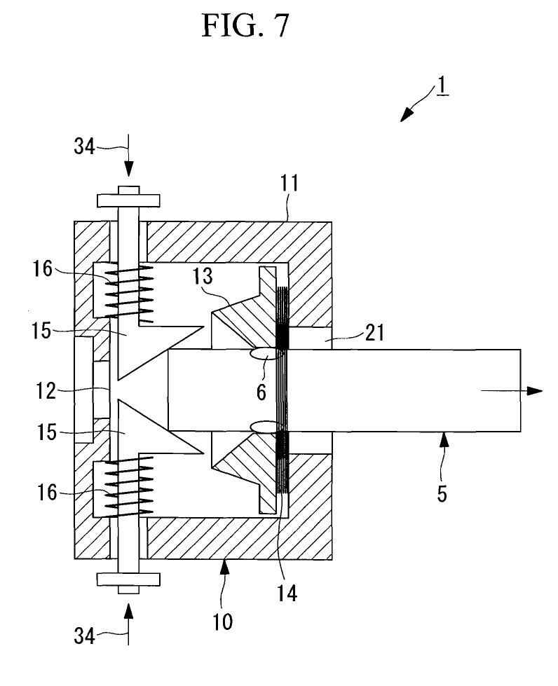

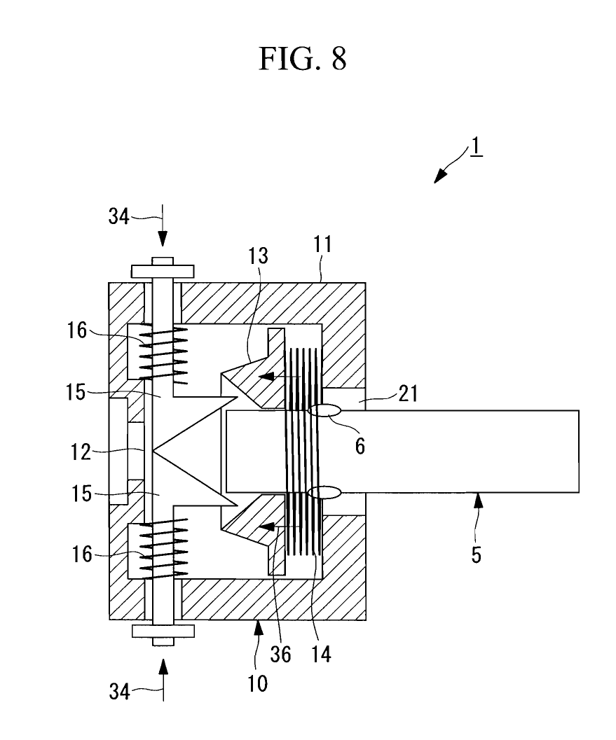

FIGS. 6 to 8 show states during removal of the plug 5 from the socket 10. FIG. 6 shows a state before the engaging projections 6 pass through the through-hole in the pressing member 13, FIG. 7 shows a state in which the engaging projections 6 are passing through the through-hole in the pressing member 13, and FIG. 8 shows a state after the engaging projections 6 have passed through the through-hole in the pressing member 13.

First, as shown in FIG. 6, when the plug 5 is pulled away from the socket 10, the engaging projections 6 move the pressing member 13 in the direction in which the plug 5 is pulled, i.e., the direction shown by an arrow 35.

Then, as shown in FIG. 7, when the plug 5 is pulled farther away from the socket 10, the pressing member 13 pushes the engaging projections 6 provided around the outer circumferential surface of the plug 5 radially inward into the plug 5.

Then, as shown in FIG. 8, when the plug 5 is pulled farther away from the socket 10, contact between the pressing member 13, the stopper members 15, and the plug 5 is released. As a result, the pressing member 13 and the stopper members 15 can be returned to the state before the plug 5 is inserted into the socket 10, i.e., to a position where the pressing member 13 is prevented from moving toward the connection surface 12, by the urging force of the coil spring 14 and the coil springs 16, respectively, as shown by the arrows 34 and 36.

As has been described, the coupling 1 according to this embodiment enables the plug 5 and the socket 10 to be easily connected and separated, and enables the end surface 7 of the plug 5 to be securely abutted against the connection surface 12 of the socket 10 when the plug 5 and the socket 10 are connected.

### Example Application

An example in which the coupling 1 according to this embodiment is applied to an endoscope system will be described below.

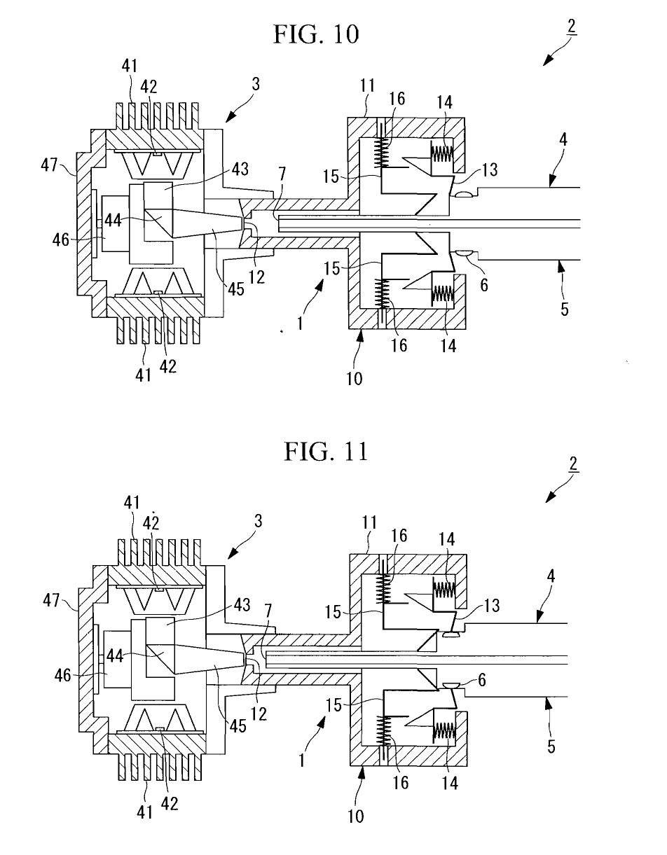

FIG. 10 schematically shows the structure of an endoscope system 2 according to this example application.

As shown in FIG. 10, the endoscope system 2 includes the coupling 1, a light source device 3 that emits illumination light, and a scope 4 that can be attached to and separated from the light source device 3 and that guides the illumination light from the light source device 3 to an observation area.

The socket 10 of the coupling 1 is provided on the light source device 3, and the plug 5 of the coupling 1 is an optical fiber (light guide member) provided in the scope 4. The connection surface 12 of the socket 10 is an emitting surface of the illumination light from the light source device 3, and the end surface 7 of the plug 5 is an incident surface of the scope 4.

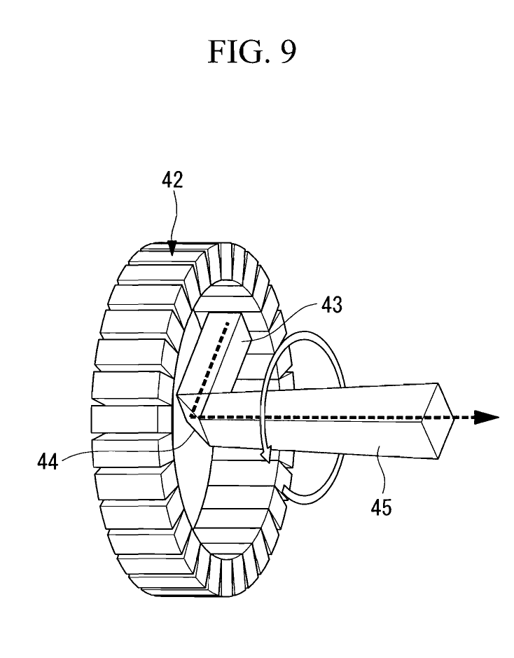

FIG. 9 is a schematic view of the main part of the light source device 3.

As show in FIGS. 9 and 10, the light source device 3 includes a substantially cylindrical radiator 41, a plurality of light sources 42 that are arranged at intervals around the inner circumferential surface of the radiator 41 and emit illumination light in the inward radial direction of the radiator 41, a light guide rod 43 that guides the illumination light emitted from the light sources 42, a reflecting member 44 that reflects the illumination light guided to the light guide rod 43 in the axial direction of the radiator 41, an illumination rod 45 that guides the illumination light reflected by the reflecting member 44, a motor 46 that rotates and drives the light guide rod 43, the reflecting member 44, and the illumination rod 45, and a motor base 47 to which the motor 46 is fixed.

The radiator 41 releases the heat generated by the light sources 42 outside.

The plurality of light sources 42, for example, LEDs (semiconductor light sources), are arranged in a circle such that their optical axes extend toward the axis of the radiator 41. By sequentially lighting the light sources 42 in a pulsed manner, they instantaneously emit high-intensity illumination light.

The motor 46 synchronizes the lighting cycle of the light sources 42 and the rotating cycle to allow the illumination light emitted from the light sources 42 to be incident on the light guide rod 43. Thus, the high-intensity illumination light emitted by lighting the light sources 42 in a pulsed manner is continuously emitted in the axial direction of the radiator 41.

In the endoscope system 2 having the above-described structure, the operation of connecting the light source device 3 and the scope 4 will be described below with reference to FIGS. 10 to 13.

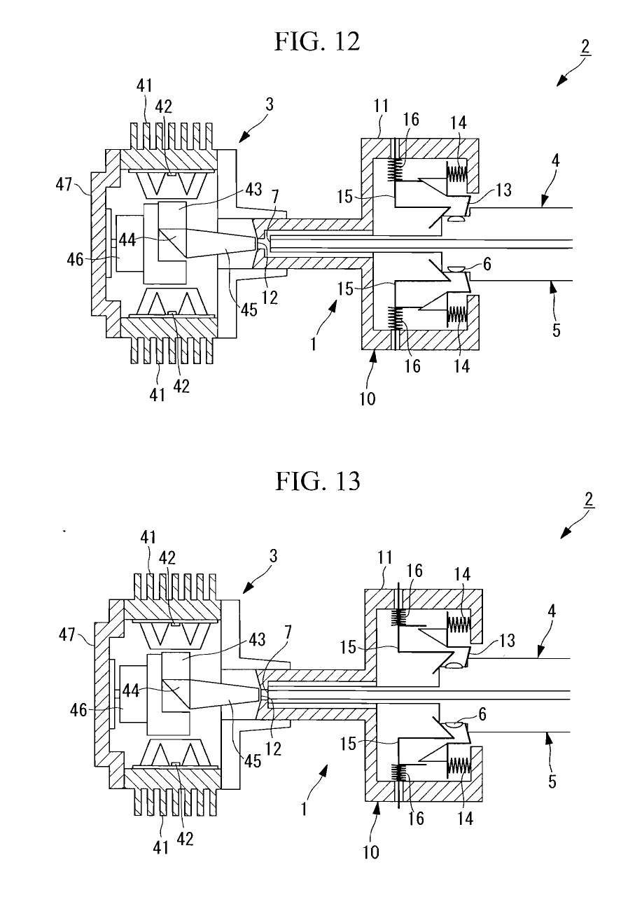

FIGS. 10 to 13 show states during connection of the light source device 3 and the scope 4. FIG. 10 shows a state before the engaging projections 6 pass through the through-hole in the pressing member 13, FIG. 11 shows a state in which the engaging projections 6 are passing through the through-hole in the pressing member 13, and FIG. 12 shows a state after the engaging projections 6 have passed through the through-hole in the pressing member 13. FIG. 13 shows a state in which the light source device 3 and the scope 4 are completely connected.

First, as shown in FIG. 10, when the plug 5 provided in the scope 4 is inserted into the socket 10 provided on the light source device 3, the stopper members 15 are moved radially outward.

Next, as shown in FIG. 11, when the plug 5 is deeply inserted into the socket 10, the stopper members 15 stop the pressing member 13 from moving.

Then, as shown in FIG. 12, when the plug 5 is more deeply inserted into the socket 10, the engaging projections 6 pass through the through-hole in the pressing member 13.

Finally, as shown in FIG. 13, the stopper members 15 no longer stop the pressing member 13, and the urging force of the coil spring 14 is transmitted to the plug 5 through the pressing member 13 and the engaging projections 6. Thus, the end surface 7 of the plug 5 is pressed against the connection surface 12 of the socket 10.

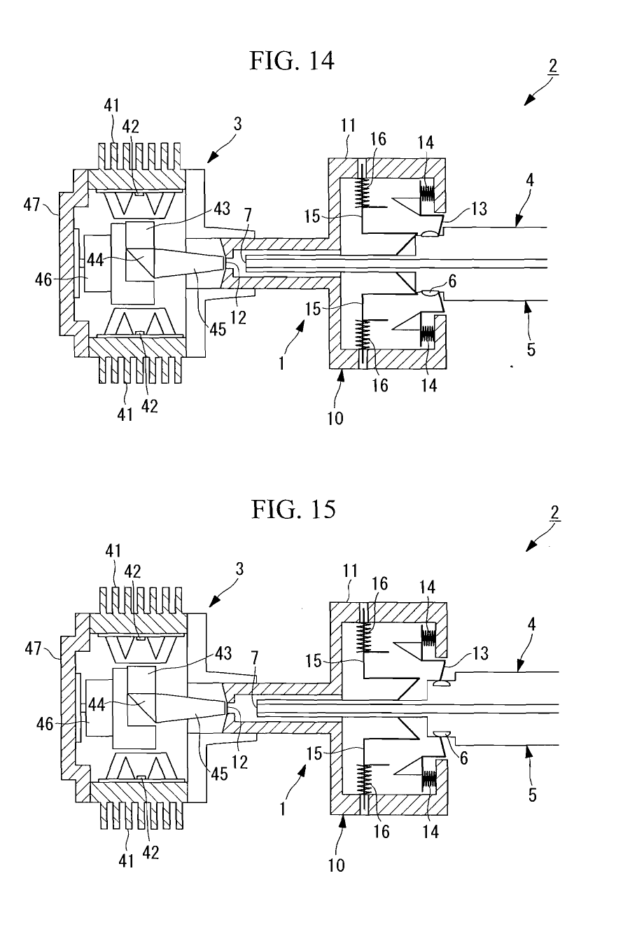

The operation of separating the light source device 3 and the scope 4 of the endoscope system 2 having the above-described structure will be described below with reference to FIGS. 14 to 16.

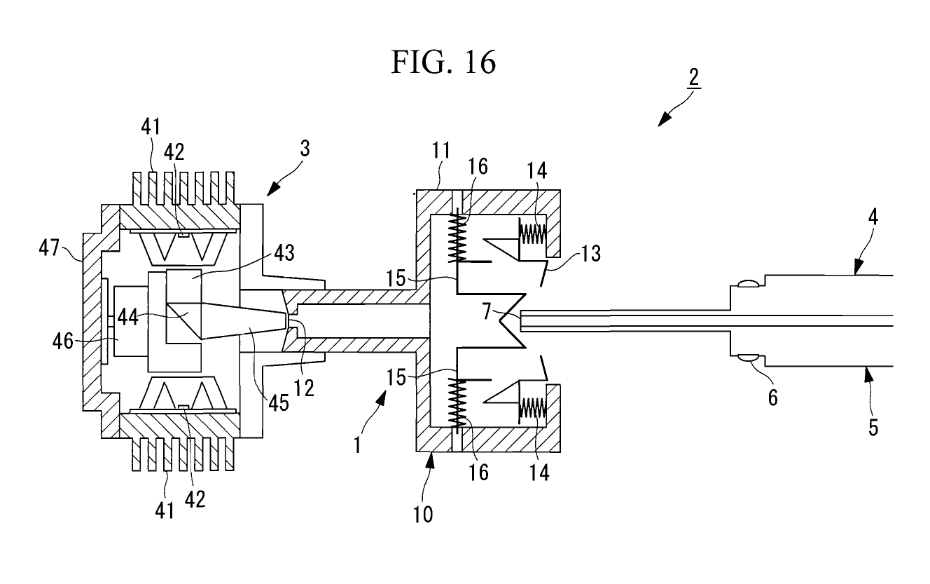

FIGS. 14 to 16 show states during separation of the light source device 3 and the scope 4. FIG. 14 shows a state before the engaging projections 6 pass through the through-hole in the pressing member 13, FIG. 15 shows a state in which the engaging projections 6 are passing through the through-hole in the pressing member 13, and FIG. 16 shows a state after the engaging projections 6 have passed through the through-hole in the pressing member 13.

First, as shown in FIG. 14, when the plug 5 provided in the scope 4 is pulled away from the socket 10 provided on the light source device 3, the engaging projections 6 move the pressing member 13 in the direction in which the plug 5 is pulled.

Then, as shown in FIG. 15, when the plug 5 is pulled farther away from the socket 10, the pressing member 13 pushes the engaging projections 6 radially inward into the plug 5.

Then, as shown in FIG. 16, when the plug 5 is pulled farther away from the socket 10, the pressing member 13 and the stopper members 15 are returned to the state before the plug 5 is inserted into the socket 10 by the urging force of the coil spring 14 and the coil springs 16, respectively.

As has been described, the endoscope system 2 according to this example application enables the scope 4 and the light source device 3 to be easily connected and separated, and enables the incident surface 7 of the scope 4 to be securely pressed against the emitting surface 12 of the illumination light from the light source device 3. Thus, stable illumination light can be emitted onto the observation area. Furthermore, the provision of the socket 10 on the light source device 3 makes connection to existing scopes possible.

Although the embodiments of the present invention have been described in detail with reference to the drawings, the specific structure is not limited to these embodiments, and it includes modifications to the design so long as they do not depart from the scope of the present invention.

For example, although the stopper members 15 have been described as being provided so as to be movable in the direction intersecting the insertion direction of the plug 5, they may of course be provided so as to be movable in a direction perpendicular to the insertion direction of the plug 5.

Although the pressing member 13 has been described as a circular member having a through-hole, it may instead be a plate-shaped or rod-shaped member with the engaging projections 6 formed in a circle.

Although the stopper members 15 have been described as rod-shaped or plate-shaped members, they may instead be circular members.

By arranging the plurality of stopper members 15 such that the ends of their inclined surfaces are in contact with one another when the plug 5 is not inserted into the socket, the stopper members 15 can be used as a shutter that prevents the light from the light source device 3 from being emitted outside.

Although the urging member of the pressing member 13 and the other urging members of the stopper members 15 have been described as the coil spring 14 and the coil springs 16, they may instead be elastic bodies such as leaf springs.

What is claimed is:

1. A coupling comprising:
a columnar plug; and
a socket into which the plug is inserted and connected, the plug having engaging projections that are radially projected from and retracted into an outer circumferential surface thereof,
the socket having
a connection surface to which an end surface of the plug is abutted;
a pressing member having a through-hole that is slightly larger than the outside diameter of the plug;
an urging member that urges the pressing member along an insertion direction of the plug toward the connection surface; and
stopper members that stop the pressing member so as to push it in a direction opposite to a direction toward the connection surface in response to the insertion of the plug into the socket until the engaging projections pass through the through-hole in the pressing member,
wherein the stopper members stop pushing the pressing member after the engaging projections pass through the through-hole in the pressing member, and the pressing member that is no longer pushed urges the plug toward the connection surface via the engaging projections.

2. The coupling according to claim 1,
wherein the stopper members are provided so as to be movable in a direction intersecting the insertion direction of the plug and have inclined surfaces with which an end of the plug is brought into contact, the inclined surfaces being inclined inward along the direction intersecting the insertion direction of the plug in a direction toward the connection surface,
wherein an edge of the end surface pushes apart the inclined surfaces facing thereto and moves the stopper members in the direction intersecting the insertion direction of the plug when the plug is inserted into the socket, and
wherein the inclined surfaces facing thereto being pushed apart urge the pressing member in a direction opposite to the direction toward the connection surface by a force greater than the urging force of the urging member.

3. The coupling according to claim 2, further comprising other urging members that urge the stopper members inward along the direction intersecting the insertion direction of the plug.

4. The coupling according to claim 2,
wherein the pressing member and the stopper members are arranged such that the end of the plug is in contact with the inclined surfaces of the stopper members before the engaging projections pass through the through-hole, and
wherein the pressing member has a tapered inner surface with which edges of the inclined surfaces of the stopper members are brought into contact, the tapered inner surface being inclined radially outward of the through-hole in the direction toward the connection surface.

5. An endoscope system comprising:
the coupling according to claim 1;
a light source device that emits illumination light; and
a scope that is provided so as to be attachable to and separable from the light source device and that guides the illumination light from the light source device to an observation area,
wherein the plug is a light guide member provided in the scope, the socket is provided in the light source device, the connection surface is an emitting surface of the illumination light from the light source device, and the end surface of the plug is an incident surface of the light guide member.
